(19) United States
(12) Patent Application Publication
(10) Pub. No.: US 2006/0045882 A1
(43) Pub. Date: Mar. 2, 2006

(54) Developing oral tolerance for airborne allergens

(76) Inventor: Pradeep Shrikrishna Limaye, Westfield, NJ (US)

Correspondence Address: Pradeep S. Limaye, 3 Azalea Trail, Westfield, NJ, 07090, US

(21) Appl. No.: 10/925,846

(22) Filed: Aug. 26, 2004

(51) Int. Cl. A61K 39/38; A61K 39/00

(52) U.S. Cl. 424/184.1

(57) ABSTRACT

A method is disclosed for developing desensitization to airborne allergens in persons suffering from allergic reactions to such substances. This desensitization is developed by orally administering airborne allergens collected from the environment which leads to allergy symptoms in the user. The illustrative embodiments collect airborne allergens in water by passing air containing the allergens through water or passing it past water droplets or wet surfaces.

### CROSS-REFERENCE TO RELATED APPLICATIONS

Not Applicable

### FIELD OF THE INVENTION

The present invention relates to allergies in general, and, more particularly to desensitizing the body to airborne allergens.

### BACKGROUND OF THE INVENTION

Allergies are caused by a misguided reaction to foreign substances by the immune system. The substances that trigger allergy are called allergens. Examples of airborne allergens include pollens, dust mite, molds and dander. The most common allergic conditions include hay fever (allergic rhinitis), asthma, allergic eyes (allergic conjunctivitis), allergic eczema, hives (urticaria), and allergic shock (also called anaphylaxis and anaphylactic shock).

Although many medicines are available for treating the symptoms of allergies, the only way to prevent allergic reactions is through desensitization to allergens, which is achieved by stimulation of the immune system by injecting gradually increasing doses of the substances to which a person is allergic.

The common form of desensitization therapy is “allergy shots” which use injections of allergens specific to the patient. The allergen responsible for the allergic reaction in the patient must be identified before the therapy can begin. If the allergen can be identified, this form of treatment is very effective for allergies to pollen, mites and cats. Current allergy immunotherapy usually takes 6 months to a year to become effective and injections (“shots”) are usually required for 3-5 years. This desensitization therapy is expensive and time consuming.

It is desirable that a person suffering from allergies be able to desensitize his or her body without the expense and time associated with first identifying the reaction-causing allergen and then desensitizing the body through injections.

Another approach to desensitization is through oral tolerance. It has long been believed that if enough of the allergy-causing allergen, usually a protein in pollen, dust or dander, is eaten, the body is actually vaccinated against an allergic reaction to that allergen. Oral Tolerance was first described at the turn of the 20th Century by H G Wells, the author of War of the Worlds. Animal studies have shown oral tolerance to be effective for dust mite allergies (Sato, et al., Immunology, Volume 95, Issue 2, 1998, Page 193).

A common method of desensitization to pollen via oral tolerance is to ingest local bee pollen. Many people report that their allergies improve when they take local bee pollen or honey to desensitize themselves. However, many of the pollens that cause allergies are not the ones bees pick up, and it can be difficult to find local pollen or honey.

The limitation with the method of using bee pollen is that the person who will ingest the bee pollen does not generally live where the pollen is collected. This causes the bee pollen to contain pollens that the user is not exposed to as an inhalant. Similarly, the bee pollen will not contain all the allergy causing pollens prevalent in the user's environment. Bee pollen is very non-specific to the allergy causing pollens specific to every user. Also, many airborne allergens, such as proteins from dust mites or dander are not present in the bee pollen. Sensitization to these allergens cannot be achieved by ingesting bee pollen.

It is desirable that a person suffering from allergies be able to ingest allergens collected from the environment where he or she breathes. It would be of further benefit if the collection could be done in the specific areas breathing in which leads to allergy symptoms.

### SUMMARY OF THE INVENTION

The aforementioned shortcomings in the art are addressed in the present invention by providing a method and apparatus for desensitizing a person suffering from allergies to airborne allergens. The desensitization is affected by administering airborne allergens collected specifically for ingestion by a person suffering from inhalant allergies, where the collecting is done in the same environment as the one that causes the allergic symptoms in the said person.

### OBJECTS OF THE INVENTION

Accordingly several objects and advantages of the present invention are:
a) To provide a method of desensitization that is inexpensive compared to allergy shots, painless and does not require repeated visits to a doctor's office for administration.
b) To provide a method of desensitization that includes all allergens that are present in the air, which when breathed, leads to allergic reactions.
c) To provide a method of desensitization that does not include allergens that are not present in the allergy causing air.
d) To provide a method of desensitization that does not require identification of the specific allergen causing the allergic reaction.

Other objects and advantages of the present invention are:
a) To provide a method of desensitization that is safe because it only involves ingesting of substances that are already being inhaled.
b) To provide a method of desensitization that allows easy control over the amount of allergens ingested by controlling the collection time.
c) To provide a method of desensitization that when used with short collection times and resulting low concentrations of allergens provides the benefits of homeopathic preparations.
d) To provide a method of desensitization which allows portability of the collection apparatus to indoor or outdoor locales depending on the cause of the allergies.

### BRIEF DESCRIPTION OF THE DRAWINGS

FIG. 1 shows an apparatus for collecting airborne allergens in water by bubbling air containing the allergens through water.

FIG. 2 shows an apparatus for collecting airborne allergens in water by spraying water through flowing air that contains the allergens.

FIG. 3 shows an apparatus for collecting airborne allergens in water by blowing air containing the allergens past rotating disks partially submerged in water.

### REFERENCE NUMERALS IN DRAWINGS

| | | | |
|---|---|---|---|
| 1 | Container | 2 | Water |
| 3 | Air | 4 | Air outlet |
| 5 | Air pump | 6 | Tube |
| 7 | Bubbler, diffuser | 8 | Bubbles |
| 9 | Fan | 10 | Air inlet |
| 11 | Fan motor | 12 | Water pump |
| 13 | Fountain head, sprayer | 14 | Water spray |
| 15 | Disk stack | | |

### DETAILED DESCRIPTION

The present invention uses allergens collected from the air to produce oral tolerance to said allergens. In a person suffering from allergic reactions, like hay fever, runny nose etc. to breathing air containing allergens, eating the allergen induces a suppressive type of immune response. Ingestion of allergens over time strengthens this suppressive immune response and the person is desensitized to the allergens when breathed in.

The present invention collects the allergens from the air that causes the allergy, thus making the suppressive response specific to the allergens present in that air. For example, if a person develops hay fever by being outdoors in the backyard in a particular season, collecting allergens from the backyard air in that season and ingesting them will initiate the suppressive immune response to those seasonal allergens. Such collection and ingestion could be performed wherever and whenever allergic symptoms are experienced.

By collecting all particulates suspended in air, the present invention provides for building oral tolerance to any possible airborne allergen. The present invention is not limited to pollen allergies only as is the case with ingesting bee pollen. Other allergens such as dander or dust mites are also collected by the collection apparatus to be ingested by the person suffering from allergies.

The airborne material of the same composition that is collected in the present invention is also entering the person's lungs when he breathes. Thus the collected material is known to be safe. The safety of the collected material is assured by performing the collection in the allergy sufferer's home, work place or back yard. If an area has highly polluted air with noxious or poisonous substances, material for ingestion shall not be collected in that area.

The airborne allergens collected in the present invention can be administered orally once or several times a day. By controlling the amount of time for which collection is done, the amount of allergen collected is easily controlled in the present invention. The allergy sufferer can find the amount and frequency of ingestion best suited for his or her environment for both building up and maintaining the oral tolerance.

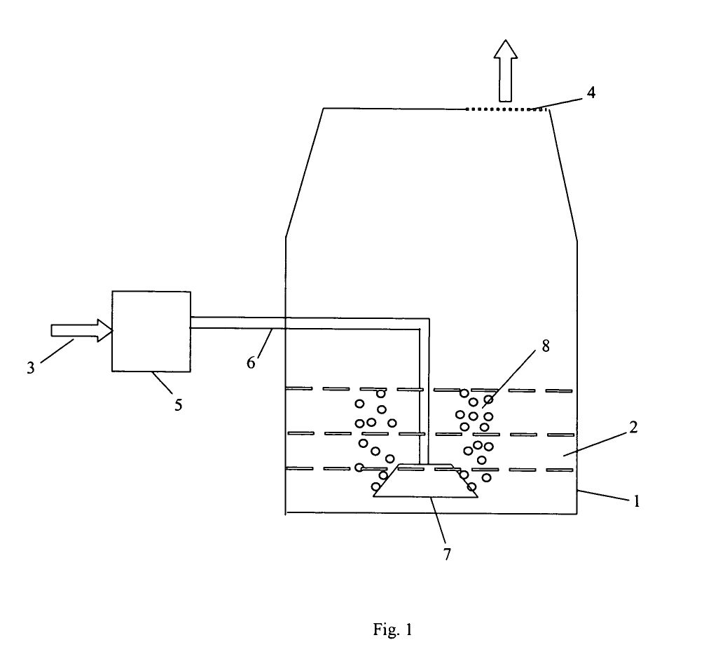

Illustrative embodiments for collecting airborne allergens in water are shown in FIGS. 1-3. In all three embodiments, water is used as the collecting medium and air containing the allergens is brought into contact with the water to scrub the allergens from the air and retain them in the water.

First illustrative embodiment for collecting the airborne allergens for ingestion is shown in FIG. 1. A container 1 holds water 2. An air pump 5 is connected by a tube 6 that enters container 1. Tube 6 is further connected to a bubbler, aerator or diffuser 7. Container 1 has an air outlet 4.

In the illustrative embodiment of FIG. 1, ambient air 3 is pumped by air pump 5 in to tube 6. Air from tube 6 flows through holes in bubbler 7 to enter water 2. Holes and air passages in bubbler 7 and tube 6 are large enough to allow all allergens to pass through into the water 2. Holes in bubbler 7 are sufficiently small to allow maximum surface area of bubbles 8. As air pulled in by pump 5 bubbles through water 2, some of the allergens in air 3 are retained in water 2. Air leaves container 1 through air outlet 4. After bubbling air through water 2 for some time, water 2 can be taken out of container 1 for ingestion of allergens retained in water 2.

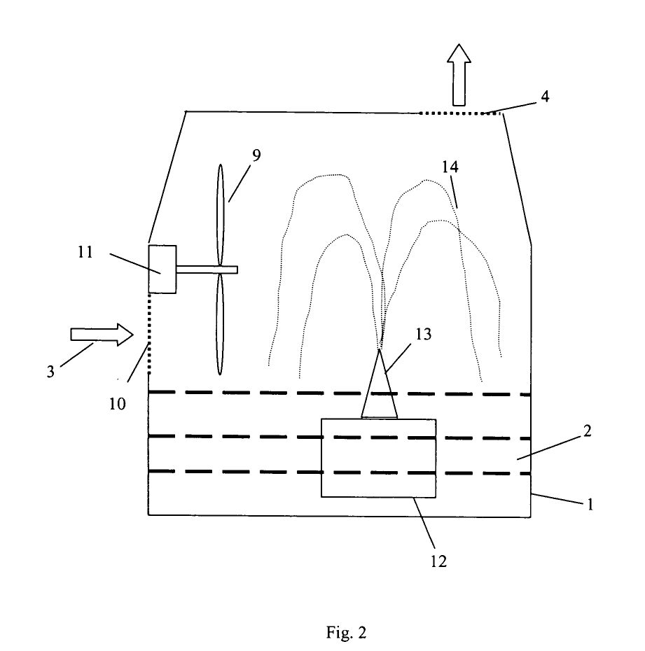

A second embodiment for collecting the airborne allergens for ingestion is shown in FIG. 2. Container 1 has air inlet 10. Container 1 further has a fan motor 11 connected to fan 9. A circulating water pump 12 sits inside container 1 and is submerged in water 2. Pump 12 is connected to a fountain head, spray head or atomizer 13.

In the embodiment of FIG. 2, pump 12 pumps water 2 through fountain head 13 to create a fountain, spray or mist 14 inside container 1. Fan motor 11 drives fan 9 to pull in ambient air 3 through air inlet 10. Air blows over fountain 14 bringing airborne allergens in contact with water. Air exits container 1 through outlet 4. After blowing air past fountain 14 for some time, water 2 can be taken out of container 1 for ingestion of allergens retained in water 2.

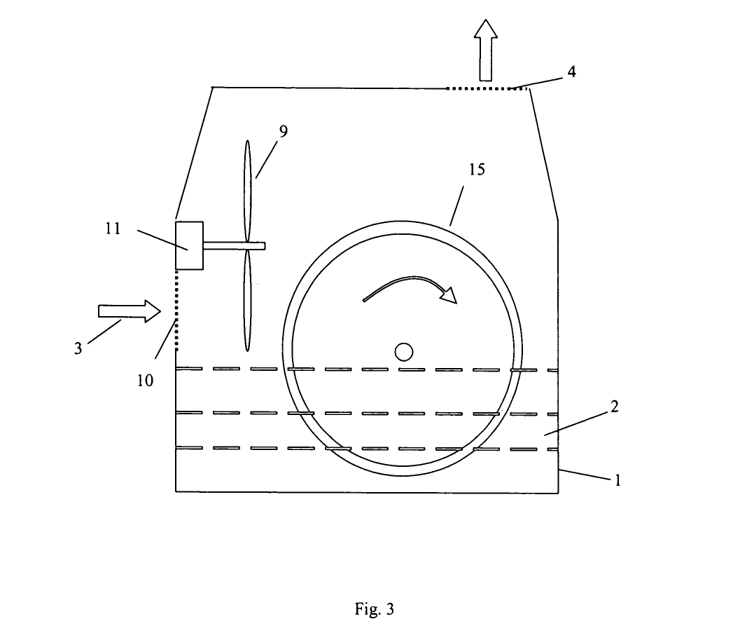

A third embodiment for collecting the airborne allergens for ingestion is shown in FIG. 3. Container 1 has a rotating stack of disks 15 partially submerged in water 2.

In the embodiment of FIG. 3, disk stack 15 has sufficient separation between its disks to allow easy air flow. Each disk picks up water as it rotates. Wet surfaces of disks in stack 15 come in contact with air 3 being blown past the disks by fan 9. Some of the allergens in the ambient air are picked up by wet surface of disks in disk stack 15 and are retained in water 2 as disk stack 15 rotates. After blowing air past rotating disk stack 15 for some time, water 2 can be taken out of container 1 for ingestion of allergens retained in water 2

### CONCLUSIONS, RAMIFICATIONS AND SCOPE OF INVENTION

The present invention provides an easy method for persons suffering from allergies to airborne allergens to desensitize themselves by collecting the airborne allergens directly from the environment where the allergy causing substances are present in the air. Ingesting these collected allergens will promote oral tolerance in the users and desensitize them.

While the above description contains many specificities, these should not be construed as limitations on the scope of the invention, but rather as exemplifications of illustrative embodiments thereof. Many other variations are possible, for example, although the illustrative embodiments utilize drinking water for collecting the airborne allergens, other embodiments of allergen collection apparatus using paper or mesh filters or electro-static precipitators are also possible, where the allergens collected on the filter or precipitator are ingested to build tolerance. The embodiments described here are for illustrative purposes only, one skilled in the art can put additives in the water to enhance adhesion between the allergens and water. Further additives can be used to kill any airborne bacteria to enhance safety. One skilled in the arts can also envision illustrative embodiment of FIG. 1 where an electric charge is imparted to the air to increase the adhesion between allergens and water or for FIG. 2 or FIG. 3 where an electric charge is imparted to the water to increase the adhesion.

Accordingly, the scope of the invention should be determined not by the embodiments illustrated, but by the appended claims and their legal equivalents.

I claim:

1. A method for developing tolerance to airborne allergens in humans allergic to said allergens, comprising
a. A means of collecting said allergens from the person's environment and
b. Orally administering collected allergens to said human.

2. The method of claim 1 wherein said means of collecting allergens comprises,
a. A means for holding water,
b. A means for bubbling the ambient air through said water to collect allergens.

3. The method of claim 1 wherein said means of collecting allergens comprises,
a. A means for holding water
b. A means for spraying said water and
c. A means for blowing the ambient air past the spray to collect airborne allergens in water droplets of said spray

4. The method of claim 1 wherein said means of collecting allergen comprises
a. A means for holding water
b. A set of disks partially submerged in said water
c. A means of rotating said disks and
d. A means for blowing the ambient air past disks to collect airborne allergens in the water wetting said disks

5. A composition for developing tolerance to airborne allergens in humans allergic to said allergens, comprising airborne particles collected from ambient air.

6. The composition of claim 5 wherein said composition includes water used for collecting said airborne particles.
